(19) United States
(12) Patent Application Publication
(10) Pub. No.: US 2012/0207685 A1
(43) Pub. Date: Aug. 16, 2012

(54) Non-ozone depleting medicinal formulations with low greenhouse effect

(75) Inventors: Julio César Vega, San Martin (AR); Fernando Toneguzzo, Burzaco (AR)

(73) Assignee: aboratorio Pablo Cassara S.R.L., Codigo (AR)

(21) Appl. No.: 13/052,054

(22) Filed: Mar. 19, 2011

Related U.S. Application Data
(63) Continuation of application No. 13/024,414, filed on Feb. 10, 2011.

(51) Int. Cl. A61K 9/12; A61P 11/08; A61P 29/00; A61P 11/00

(52) U.S. Cl. 424/45

(57) ABSTRACT

Pharmaceutical pressurized metered dose inhalers are disclosed having a composition free of CFCs and totally or partially free of HFAs, thus allowing the manufacture of medicinal aerosols without damaging the atmospheric ozone layer and with low or negligible greenhouse effect.

### FIELD OF INVENTION

The present invention relates to the formulation of metered dose inhalers that are fire of chlorofluorocarbons (CFCs) and free or partially free of hydrofluoralkanes (HFAs) and show low-greenhouse effect, good uniformity of dosing and deposition even higher than some HFA metered dose inhalers on the market.

### PRIOR ART

Inhalation has become a widely used route of administration of bronchodilators, steroids and other medications to the airways of patients suffering from respiratory diseases. One of the pharmaceutical dosage forms used for this purpose are pressurized metered dose inhalers.

Pressurized metered dose inhalers need a propellant within their formulation in order to produce a fine spray of micronized particles as the formulation is expelled through the valve stem and actuator orifice into the oral cavity of the patient. Until recently the most widely used propellants for this purpose had been chlorofluorocarbons (CFCs). However, it has been discovered that these propellants react with the ozone layer of the atmosphere and contribute to its depletion. This discovery has put enormous pressure on the companies producing and consuming these propellants around the world to reduce their production and consumption. Recently FDA and most of the governments of the world have begun to ban their use even for pressurized metered-dose inhalers.

In the past years several efforts have been made by pharmaceutical companies to re-formulate their products into non-ozone depleting formulations, mainly by replacing CFCs by Hydrofluoroalkanes (HFAs). The most widely used HFAs for this purpose have been HFA 134a (Norflurane or 1,1,1,2-Tetrafluoroethane) and HFA 227ea (1,1,1,2,3,3,3-heptafluoropropane) as can be illustrated by the teachings of Purewal et al. (U.S. Pat. No. 5,776,434), Akehurst et al. (U.S. Pat. No. 6,893,628) and others (Govind et al. U.S. Pat. No. 7,759,328). Active ingredients have been formulated in suspension (e.g. Purewal et al. U.S. Pat. No. 5,776,434, Govind et al. U.S. Pat. No. 7,759,328), solution (e.g. U.S. Pat. No. 6,045,778) and a combination of active ingredients in suspension and others in solution (e.g. patent application Ser. No. 13/024,414 filed in 2011). Several formulations with different active ingredients with the use of different excipients have been extensively studied and taught through patents. However, all this effort has been mainly aimed at reducing ozone depletion without substantially reducing greenhouse effect. This can be easily seen when ozone depletion potential and greenhouse effect of CFCs and HFAs are listed as in Table 1.

TABLE 1

| | Ozone depleting | Duration in | |
|---|---|---|---|
| | potential | atmosphere in | GWP (100 years) (**) |
| Propellant | (taking CFC as 1) | years (**) | (taking CO₂ as 1) |
| CFC 11 | 1 (*) | 45 | 4,750 |
| CFC 12 | 1 (*) | 100 | 10,900 |
| HFA 134a | 0 | 14 | 1,430 |
| HFA 227ea | 0 | 34.2 | 3,220 |

(*) Taken from www.epa.gov/Ozone/science/ods/classone.html (webpage of US Environmental Protection Agency)
(**) Taken from “http://www.ipcc.ch/pdf/assessment-report/ar4/wg1/ar4-wg1-chapter2.pdf” (2007 IPCC Fourth Assessment Report (AR4) Chapter 2, pages 212 and 213)

On the other hand, there has been only a minor effort focused on reducing both ozone depletion and greenhouse effect from pressurized metered dose inhaler. There has been a proposal to replace CFCs by using isobutane with up to 10% w/w propane in the formulation taught by Warnke in EP 0605483. This approach renders pressurized inhalers neither having any ozone depletion potential nor significant greenhouse effect as depicted in Table 2.

TABLE 2
Environmental impact of Hydrofluoroalkanes and Hydrocarbons

| | Ozone depleting | Duration in | |
|---|---|---|---|
| | potential | atmosphere in | GWP (20 years) |
| Propellant | (taking CFC as 1) | years | (taking CO₂ as 1) |
| HFA 134a | 0 | 14 | 3,830 |
| HFA 227ea | 0 | 34.2 | 5,310 |
| Propane | 0 | Months | 12 (***) |
| n-Butane | 0 | Weeks | 12 (***) |
| Isobutane | 0 | Weeks | 12 (***) |
| n-Pentane | 0 | Weeks | 12 (***) |

(***) These substances are not considered greenhouse gases by the Kyoto Protocol and it is, therefore, hard to find the exact GWP. These data are based on a publication at the NASA website “http://www.giss.nasa.gov/meetings/pollution2002/d3_edwards.html”. However, it is worth saying that in a position paper of CECED (European Committee of Manufacturers of Domestic Equipment) the value for Hydrocarbons is 3 for GWP (100 years) and it is quoted from IPCC (Intergovernmental Panel on Climate Change) Besides EPA (US Environmental Protection Agency) refers to Hydrocarbons other than methane as low GWP substances (“http://www.epa.gov/ozone/defns.html#gwp”).

However, this patent (EP 0605483) filed in 1992 has not resulted in the marketing of any commercial product and did not contain any data regarding uniformity of dosage and fine particle fraction. As Warnke teaches, formulating pressurized metered dose inhalers with hydrocarbons is full of challenges, particularly in the case of isobutane. These challenges as depicted by Warnke are:
Relatively low density in comparison with CFCs, which accelerates sedimentation and tends to make the suspensions physically unstable and could bring about problems regarding uniformity of the amount of drug delivered per actuation.
Relatively low vapor pressure at room temperature in the case of isobutane (3.2 bar at 21° C. according to USP monograph), which tends to lower the deposition of these MDIs as compared with those formulated with CFCs (CFC 11+CFC12 30/70 blend 4.5 bar at 20° C. (according to Handbook of Pharmaceutical Excipients 2^{nd}. Edition, edited by American Pharmaceutical Association & The Pharmaceutical Press, printed in Great Britain in 1994) or HFAs (Norflurane or HFA 134a: 5.7 bar at 20° C. according to Handbook of Pharmaceutical Excipients 2^{nd}. Edition, edited by American Pharmaceutical Association & The Pharmaceutical Press, printed in Great Britain in 1994). Deposition is measured in vitro using apparatuses called “impactors”. The USP allows the use of several impactors. Andersen Cascade Impactor is one of them and is extensively used. Deposition is measured as the mass of fine particles per actuation. These fine particles are those capable of reaching the bronchi and lungs when inhaling. Therefore, the larger the mass of fine particles per actuation, the larger the expected effect on the respiratory airways. Larger mass of fine particles could also allow to reduce the total amount of pharmaceutically active ingredient delivered to the patient to get the same dose of respirable particles, thus improving therapeutic efficacy by reducing the systemic exposure and, therefore, the potential adverse effects.
Flammability of the propellants make it difficult to stir mixtures of them at room temperature.

Even though Warnke says that he thought to have overcome these difficulties, suspensions taught by him have not been submitted to performance tests with metering valves or even filled into cans fitted with valves to evaluate their performance characteristics. Furthermore, Warnke only teaches formulations having only active ingredient, a surfactant and isobutane with a maximum of 10% propane in the formulation. In the present invention other formulation compositions have been found to render useful formulations for pharmaceutical metered dose inhalers.

Therefore, even though hydrocarbons have been tested as possible substitute propellants for pressurized metered dose inhalers, suitable formulations to get adequate performance characteristics for medicinal pressurized metered dose inhalers remain to be found.

### DESCRIPTION OF THE INVENTION

Surprisingly, the inclusion of an alcohol allows formulating suspension and solution metered dose inhalers using hydrocarbons as propellants with the same or higher deposition that some hydrofluoroalkane formulations, achieving satisfactory uniformity of dose.

### DETAILED DESCRIPTION OF THE INVENTION

It has been surprisingly discovered that the addition of an alcohol can achieve substantially more stable suspension formulations based on hydrocarbon propellants, with adequate uniformity of dose and higher deposition than some hydrofluoroalkane formulations on the market. The said formulations contain hydrocarbons as main propellants and may include a certain proportion of hydrofluoroalkanes, as well. At the same time the addition of alcohol enables the use of two-step pressure filling, which is much safer, because the first portion to be filled is a concentrate of dissolved or suspended active ingredient in ethanol or other suitable alcohol or mixtures thereof. Addition of alcohol also allows to dissolve some active ingredients such as ipratropium bromide, fenoterol hydrobromide and others to have solution formulations of these active ingredients stabilized by the addition of acids.

While not wishing to be limited to any particular theory, it is believed that addition of alcohol to the formulation helps to achieve the foregoing results because it deeply changes the dielectric constant of the dispersing medium, thus modifying flocculation behavior and floc size. This change is so profound that even the addition of n-Propane in large proportion does alter the flocculation behavior of the suspended particles.

In all embodiments the propellants used are hydrocarbons or suitable blends of hydrocarbons with hydrofluoroalkanes. Hydrofluoroalkanes can be selected from the group: Norflurane (1,1,1,2-Tetrafluoroethane, also called HFA 134a), 227ea heptafluoropropane) or others known in the art. Hydrocarbons can be selected from the group: Isobutane, Propane, n-Butane, n-Pentane or others known in the art.

In all embodiments the alcohol used is one from the group: Ethanol anhydrous, Isopropanol and others known in the art. The amount to be used should be between 0.1 to 20% w/w in the case of formulations containing at least one suspended active ingredients and 1 to 30% w/w in the case of formulations containing only dissolved active ingredients. Water may be added if needed.

In all embodiments a suitable amount of a pharmaceutically active ingredient is added to render the correct dose when a puff is released from the valve metering chamber. Active ingredients could be: Salbutamol, Salbutamol Sulfate, R-Salbutamol and its salts, Beclometasone Dipropionate, Budesonide, Fluticasone Propionate, Fluticasone Fumarate, Salmeterol Xinafoate, Formoterol Fumarate Dihydrate, Fenoterol Hydrobromide, Ipratropium Bromide, Ciclesonide and other salts and derivates as well as other therapeutically active substances suitable to be administered by inhalation.

In some embodiments, particularly in those where at least one active ingredient is suspended, suspension stabilizers or surfactants are included and taken from the group: Oleic acid, Sorbitan Trioleate, Lecithin, perfluorinated surfactants, polyethyleneglycols, poloxamers, polyvinylpyrrolidone or others known in the art. The amount used is between 0.001 and 5% w/w depending on the active ingredient and stabilizer used.

In some embodiments active ingredients are suspended and in some others they are dissolved using a suitable amount of an alcohol or an alcohol and water.

In those embodiments where the active ingredient is suspended, it should be micronized so that 100% of the particles lie below 20 μm and 95% of the particles lie below 10 μm.

In some embodiments, particularly in the case of formulations having at least one dissolved pharmaceutically active ingredient, a stabilizer such as an acid and/or other antioxidants is helpful. Acids can be taken from the group: Citric Acid, Tartaric Acid, EDTA, Hydrochloric Acid, Sulfuric Acid and others known in the art. Antioxidants can be taken from the group: Ascorbic Acid, Tocopherol, Tocopherol Acetate, EDTA, their salts and/or derivatives and others known in the art. The inclusion of alcohol raises the solubility of these excipients and allows their introduction when needed.

In all embodiments the formulation is packaged into cans fitted with a metering valve.

### BRIEF DESCRIPTION OF DRAWING

FIG. 1. Flocculation behavior of several formulations using hydrocarbons as propellants, with and without ethanol having budesonide as suspended active ingredient and with inactive ingredients as shown in Example 1. The left image shows the initial condition and the right image depicts the image of formulation after ten seconds.

FIG. 2. Flocculation behavior of several formulations using hydrocarbons as propellants, with and without ethanol having Salbutamol Sulfate as suspended active ingredient and Ipratropium Bromide Monohydrate or Beclometasone Dipropionate as dissolved active ingredient and with inactive ingredients as shown in Example 3. The left image shows the initial condition and the right image depicts the image of formulation after ten seconds.

FIG. 3. Flocculation behavior of formulation using a hydrocarbon and a hydrofluorocarbon as propellants, with ethanol having Salbutamol Sulfate as suspended active ingredient and with inactive ingredients as shown in Example 4. The left image shows the initial condition and the right image depicts the image of formulation after ten seconds.

### EXAMPLE 1

This example illustrates the flocculation behavior of several formulations using hydrocarbons as propellants, with and without ethanol having budesonide as suspended active ingredient.

Composition w/w

| Ingredient | A | B | C | D |
|---|---|---|---|---|
| Budesonide | 0.73 | 0.73 | 0.65 | 0.73 |
| Sorbitan Trioleate | 0.65 | 1.31 | 0.64 | 0.65 |
| Ethanol anhydrous | — | — | 5 | 2 |
| Isobutane qs | qs 100 | qs 100 | qs 100 | qs 100 |

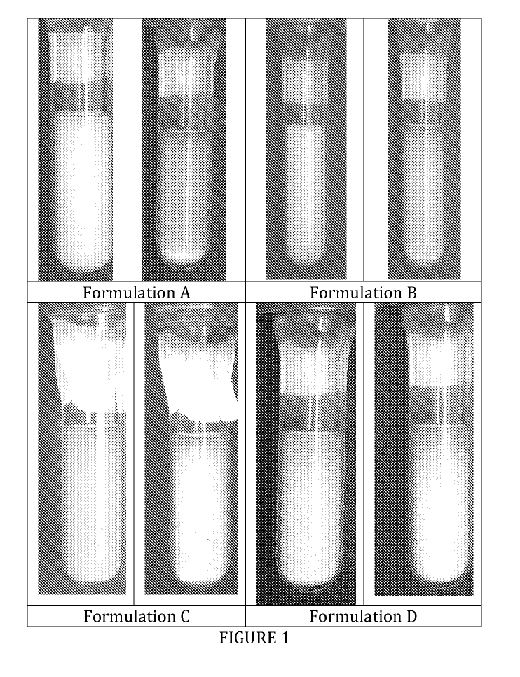

The formulations were packaged into pressurized glass test tubes and photographed (see FIG. 1). Formulations C and D clearly presents flocculation without quick sedimentation. This is advantageous because flocs are loose aggregates linked by relatively weak electrostatic forces. This is the best way to avoid “caking”, which is the formation of tightly aggregated sediment very difficult to re-disperse. In the photographs taken it is evident that the formulations without Ethanol do not flocculate and tend to form sediment very quickly at the bottom of the test tubes.

Photographs of formulations A, B, C and D at time zero and after 10 seconds are depicted in order to see the presence or absence of flocculation and formation of a tight sediment in alcohol-free formulations.

### FIG. 1

### EXAMPLE 2

This example illustrates formulations with dissolved active ingredients.

Composition w/w

| Ingredient | A | B |
|---|---|---|
| Ipratropium Bromide Monohydrate | 0.07 | — |
| Beclometasone Dipropionate | — | 0.18 |
| Ethanol | 10 | 5 |
| Isobutane | qs 100 | qs 100 |

### EXAMPLE 3

This example illustrates formulations with suspended and dissolved active ingredients.

Composition as w/w

| Ingredient | A | B |
|---|---|---|
| Salbutamol Sulfate | 0.43 | 0.43 |
| Ipratropium Bromide Monohydrate | 0.07 | — |
| Beclometasone Dipropionate | — | 0.18 |
| Sorbitan Trioleate | 1.3 | — |
| Lecithin | — | 0.5 |
| Ethanol | 10 | 5 |
| Isobutane | qs 100 | qs 100 |

In both formulations Salbutamol Sulfate is suspended and the other pharmaceutically active ingredient is dissolved.

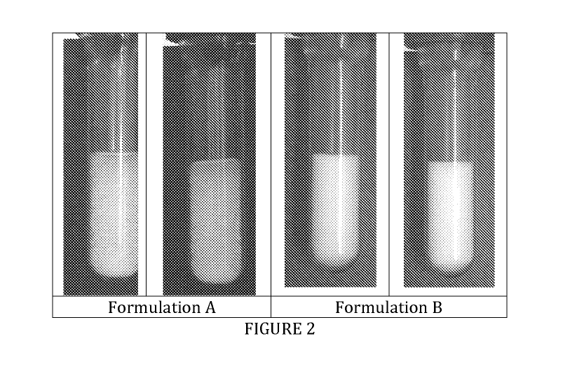

Images reveal an excellent sedimentation behavior without forming of tight sediment at the bottom of the test tube (see FIG. 2).

### FIG. 2

### EXAMPLE 4

This example illustrates the possibility of adding hydrofluoroalkane as additional propellant in a formulation.

| Ingredient | % w/w |
|---|---|
| Salbutamol Sulfate | 0.18 |
| Sorbitan Trioleate | 0.30 |
| Ethanol | 2.0 |
| HFA 134a | 29.4 |
| Isobutane | qs 100 |

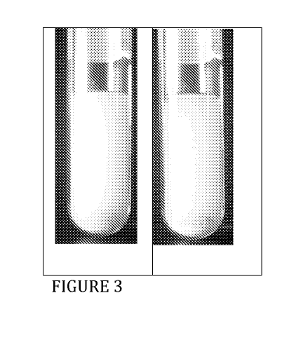

The following image illustrates the slow sedimentation behavior of this formulation: (see FIG. 3)

### FIG. 3

### EXAMPLE 5

This example illustrates the performance characteristics of a Salbutamol Sulfate formulation packaged into cans fitted with a valve.

| Ingredient | mg/actuation |
|---|---|
| Salbutamol Sulfate | 0.120 |
| Sorbitan Trioleate | 0.180 |
| Ethanol anhydrous | 0.554 |
| Isobutane qs | qs 27.7 |

The ethanolic concentrate was filled into the cans, valve crimped later onto them and finally Isobutane was filled under pressure through the valve.

The pressurized metered dose inhalers were tested for uniformity of delivered dose using USP European Pharmacopeia sampling apparatus and the range of percent of mean value was minimum 87.8% to maximum 104.6%. [The pharmacopeia requirements is that 9 out of 10 shots should be within 75-125% and 1 shot should be within 65 and 135%.]

Fine particle mass was determined using Andersen Cascade Impactor and found to be 51.2 μg per actuation.

From literature (Dellamary L A et al., Pharmaceutical Research Vol. 17, No. 2, 200, pages 168-174) we know that Proventil HFA presently on the market in the US has a fine particle mass per shot around 45.1 μg.

As can be seen this formulation surprisingly achieves a higher mass of fine particles with the same amount of Salbutamol Sulfate per actuation (in both cases 0.120 mg per actuation), even though the vapor pressure of Isobutane (3.2 bar at 21° C. according to USP monograph) is much lower than that of Norflurane (propellant used in Proventil HFA formulation and having a vapor pressure of 5.7 bar at 20° C. according to Handbook of Pharmaceutical Excipients 2^{nd}. Edition, edited by American Pharmaceutical Association & The Pharmaceutical Press, printed in Great Britain in 1994).

### EXAMPLE 6

This example illustrates the performance characteristics of another Salbutamol Sulfate formulation containing Propane and Isobutane as propellants packaged into cans fitted with a valve.

| Ingredient | mg/actuation |
|---|---|
| Salbutamol Sulfate | 0.120 |
| Sorbitan Trioleate | 0.180 |
| Ethanol anhydrous | 0.554 |
| Isobutane | 18.8 |
| Propane qs | qs 27.7 |

The ethanolic concentrate was filled into the cans, valve crimped later onto them and finally Isobutane was filled under pressure through the valve.

The pressurized metered dose inhalers were tested for uniformity of delivered dose using USP European Pharmacopeia sampling apparatus and the range for percent of mean value was minimum 83.9% and maximum 110.7% [The pharmacopeial requirements is that 9 out of 10 shots should be within 75-125% and 1 shot should be within 65 and 135%.]

Fine particle mass was determined using Andersen Cascade Impactor and found to be 61.2 μg per actuation.

From literature (Dellamary L A et al., Pharmaceutical Research Vol. 17, No. 2, 200, pages 168-174) we know that Proventil HFA presently on the market in the US has a fine particle mass per shot around 45.1 μg.

As can be seen this formulation surprisingly achieves a higher mass of fine particles (ca. 36% more) with the same amount of Salbutamol Sulfate per actuation (in both cases 0.120 mg per actuation), even though the vapor pressure of the mixture Propane+Isobutane 30/70% w/w used in this formulation (5.1 bar according to the means values of vapor pressure acceptable range for Propane and Isobutane at 21° C. according to USP) is lower than that of Norflurane (propellant used in Proventil HFA formulation having a vapor pressure of 5.7 bar at 20° C. according to Handbook of Pharmaceutical Excipients 2^{nd}. Edition, edited by American Pharmaceutical Association & The Pharmaceutical Press, printed in Great Britain in 1994).

1. A corrosion-resistant pharmaceutical pressurized metered dose inhaler comprising:
at least one dissolved pharmaceutically active ingredient;
at least one cosolvent;
at least one suspended pharmaceutically active ingredient having a polar group;
at least one dissolved halide compound;
at least one propellants;
at least one aliphatic alcohol between 0.1 and 30% w/w;
water comprising from 0.1% to 10% w/w;
an acid comprising 0.000001% to 0.01 w/w;
at least one surfactant between 0.001 and 5% w/w;
capable of delivering between 20 and 200 uL of formulation per actuation.

2. The pharmaceutical pressurized metered dose inhaler of claim 1 wherein the hydrocarbon propellant comprises up to the amount of 30% w/w of the contents of said pharmaceutical pressurized metered dose inhaler.

3. The pharmaceutical pressurized metered dose inhaler of claim 1 wherein said active ingredient is in a suspension form.

4. The pharmaceutical pressurized metered dose inhaler of claim 1 wherein said active ingredient is in a solution form.

5. The pharmaceutical pressurized metered dose inhaler of claim 1 wherein said surfactant is sorbitan trioleate.

6. The pharmaceutical pressurized metered dose inhaler of claim 1 wherein said aliphatic alcohol is ethanol anhydrous.

7. The pharmaceutical pressurized metered dose inhaler of claim 1 where said inhaler additionally includes an antioxidant selected from a group of antioxidants consisting of tocopherol, tocopherol acetate ascorbic acid and EDTA and its salts.

8. The pharmaceutical pressurized metered dose inhaler of claim 1 where said inhaler additionally includes an organic acid or an inorganic acid in a concentration ranging from 0.00001 to 0.1% w/w to reduce chemical degradation.

9. The pharmaceutical pressurized metered dose inhaler of claim 1 wherein said metering valve delivers 50-100 uL of formulation per actuation.

10. The pharmaceutical pressurized metered dose inhaler of claim 1 wherein said active ingredient is selected from a group of drugs administered by inhalation consisting of salbutamol, salbutamol sulfate, beclomethasone dipropionate, budesonide, formoterol fumarate, fluticasone propionate, fluticasone fumarate, mometasone furoate, salmeterol xinafoate, ciclesonide, ipratropium bromide, oxitropium bromide, and tiotropium bromide.

11. The pharmaceutical pressurized metered dose inhaler of claim 1 wherein said propellant is derived from hydrocarbons containing isobutane, butane, or isopropyl groups.
